(19) United States
(12) Patent Application Publication
(10) Pub. No.: US 2026/0331280 A1
(43) Pub. Date: Oct. 22, 2026

(54) CARE RECIPIENT STATUS NOTIFICATION DEVICE

(71) Applicant: Brian Skuse, Boston, MA (US)

(72) Inventor: Brian Skuse, Boston, MA (US)

(73) Assignee: Brian Skuse, Boston, MA (US)

(21) Appl. No.: 19/730,525

(22) Filed: Jul. 2, 2026

Related U.S. Application Data
(63) Continuation-in-part of application No. 18/956,753, filed on Nov. 22, 2024, now abandoned.
(60) Provisional application No. 63/602,154, filed on Nov. 22, 2023.

(51) Int. Cl. G16H 40/67

(52) U.S. Cl. CPC G16H 40/67

(57) ABSTRACT

Embodiments of the instant disclosure relate to a care recipient status notification (CRSN) device. The CRSN device includes input buttons, a communications device, and a control circuit communicatively coupled to each input button and the communications device. The CRSN device is registered to and used by a care recipient. Each input button is associated with an input signal that is unique to that particular input button. Each input signal is associated with a unique status message. The control circuit causes a server to transmit the status message to a computing device when the input signal is received. The status message is a text message. The server transmit data via a cellular network protocol. Each input button sends a signal to the control circuit that translates it to an operational command to cause the server to transmit the status message to the computing device when pressed.

### CROSS REFERENCE TO RELATED APPLICATIONS

This application is a continuation-in-part of U.S. Appl. Ser. No. 18/956,753, filed Nov. 22, 2024, which claims benefit of U.S. Provisional Appl. Ser. No. 63/602,154, filed Nov. 22, 2023, the contents of which are incorporated by reference herein in their entirety.

### TECHNICAL FIELD

The instant disclosure generally relates to telecommunications devices, and more specifically, to telecommunications devices for requesting care services.

### BACKGROUND

Care services are services for adults that encompass a range of non-medical and personal support designed to help adults of advanced age (e.g., at least 65 years old) and/or infirmed adults maintain their independence, health, and quality of life. These services range from in-home companion care to assisted living. For example, as individuals age, complex health conditions and mobility issues can make it difficult to manage everyday activities independently. Care services are crucial because they promote independence, manage health and safety, nurture well-being, and/or provide family support.

Elder care can be tailored to an individual's unique physical, mental, and emotional health needs. Common types of care can include personal care, home health care, companion and homemaker services, and residential care. Personal care can involve assistance with daily tasks such as bathing, dressing, grooming, and mobility. Home health care can refer to skilled services provided at home, including nursing care, medication management, physical therapy, and chronic disease management. Companion and homemaker services may offer support with tasks like grocery shopping, meal preparation, and light housekeeping, along with social interaction to prevent isolation. Residential care can include facilities like assisted living communities or nursing homes that provide 24/7 supervision, security, and daily support.

In the past, communication devices for care services have typically been limited to a single, basic "call button," where the care recipient (i.e., the person requesting the care service task) presses a single button to notify the caregiver (i.e., the person selected by the care recipient to perform one or more care service tasks). However, such notifications typically do not provide information regarding the nature of the alert or the associated level of urgency of the status message and fail to limit unnecessary communications between the caregiver and the care recipient. Consumers would benefit from solutions that reduce unnecessary overcommunication between the caregiver and care recipient.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

In one general aspect, a care recipient status notification (CRSN) device may include an enclosure that may include a plurality of input buttons, a communications device, and a control circuit communicatively coupled to each input button and the communications device. The CRSN device is registered to and used by a care recipient. Each input button is associated with an input signal that is unique to that particular input button. Each input signal is associated with a unique status message. The control circuit causes a server to transmit the status message to a computing device when the input signal is received. The status message is a text message. The server transmit data via a cellular network protocol.

Each input button sends a signal to the control circuit that translates it to an operational command to cause the server to transmit the status message to the computing device when pressed. In response to receiving the signal, the control circuit transmits, via the communication device, the operational command to the server to thereby cause the server to transmit the status message to the computing device. The status message reflects a status of the care recipient and includes one or more of a pain management assistance request, an physical comfort assistance request, a hydration assistance request, and a toileting assistance request. The computing device is associated with a non-medical person selected by the care recipient to perform a non-medical task for the care recipient; and the non-medical task is associated with the status message. The communications device communicates with the server via a cellular network communications protocol. The control circuit receives a first confirmation signal from the server responsive to the server receiving, from the computing device, a second confirmation signal indicating performance of the non-medical task. The server transmits the status message to a second computing device upon expiration of a predetermined time period during which the second confirmation signal is not received. Implementations of the described techniques may include hardware, a method or process, or a computer tangible medium.

In other aspects, a system for notifying a status of a care recipient is disclosed. The system includes a CRSN device, a care recipient may also include a care giver computing device, and a server communicatively coupled to the CRSN device and the care giver computing device. The server communicates via a cellular network protocol; is registered to and used by a specific care recipient; only capable of communicating with the server; includes input buttons each associated with an unique input signal. Each input signal is associated with an unique status message. The CRSN device causes the server to transmit the status message to the care giver computing device when it receives the input signal. The status message is a text message. Other embodiments of this aspect include corresponding computer systems, apparatus, and computer programs recorded on one or more computer storage devices.

### BRIEF DESCRIPTION OF THE DRAWINGS

Aspects of the present disclosure are illustrated by way of example and are not limited by the accompanying figures for which like references indicate like elements.

FIG. 1 depicts one example of a simplified communications device, in accordance with some embodiments of the instant disclosure;

FIG. 2 is a flow diagram for one example of a method of communicating a status of a care recipient using the simplified communications device of FIG. 1, in accordance with other embodiments of the instant disclosure;

FIG. 3 depicts one example of requesting an application for the simplified communications device of FIG. 1 by a care recipient, in accordance with certain embodiments of the instant disclosure;

FIG. 4 depicts one example of the care recipient of FIG. 3 pressing one of the keys on the simplified communications device, in accordance with yet still other embodiments of the instant disclosure;

FIG. 5 depicts one example of a caregiver receiving on their computing device a status of the care recipient in response to the care recipient pressing the key of FIG. 4, in accordance with some embodiments of the instant disclosure; and

FIG. 6 depicts one example of a computing device, in accordance with other embodiments of the instant disclosure.

FIG. 7 depicts one example of a printed circuit board assembly for the simplified communications device of FIG. 1, in accordance with certain embodiments of the instant disclosure.

FIG. 8A is a block diagram illustrating a status message data processing environment, in accordance with some embodiments of the instant disclosure.

FIG. 8B is a block diagram illustrating the components of the care recipient status notification device of FIG. 1, in accordance with certain embodiments of the instant disclosure.

FIG. 9 is a block diagram illustrating the information flow of the status message data processing environment of FIG. 6, in accordance with other embodiments of the instant disclosure.

FIG. 10 depicts a block diagram of components of the server of FIG. 8A executing a caregiver program, in accordance with certain embodiments of the present disclosure.

Corresponding reference characters indicate corresponding parts throughout the several views. The embodiments set out herein are only examples and illustrate currently preferred embodiments of the instant disclosure, and such examples are not to be construed as limiting the scope of the invention in any manner.

### DETAILED DESCRIPTION

As used herein, a "simplified communications device" refers to a limited-purpose electronic communications device with two or more keys, each of the keys corresponding to a different status message of a person using the simplified communications device, the simplified communications device relying on a wirelessly connected computing device and an application on the computing device to automatically send a status message when one of the keys is pressed.

As used herein, the term "key" or "keys" refers to keys, buttons or the like on a simplified communications device, for example, one or more capacitive touch screens, or, as another example, physical keys that can be pushed and doing so causes a signal indicative of a status of a care recipient to be sent out from the simplified communication device to a computing device associated with the care recipient.

As used herein, the term "computing device" or "computing unit" refers to a computing device with one or more processors, memory and one or more applications stored in the memory. For example, the applications can include an operating system, a photo application, messaging application and/or other general or special purpose applications. Examples of computing devices include desktop computers, laptop computers, smart phones, tablets, tablet computers, small form factor computers, etc. In addition, such computing devices may be accompanied by one or more peripheral devices. For example, peripheral devices may include a pen or other pointing device, a keyboard, a monitor, etc.

As used herein, the term "processor" includes "chips," central processing units (CPUs), graphics processing units (GPUs), integrated circuitry performing processing (e.g., field programmable gate arrays (FPGAs), integrated circuitry made using large-scale integration (LSI) or very large-scale integration (VLSI)), logic circuits and "systems on chip," which include most or all of the components of a computer. Processors work in conjunction with applications, apps, software, firmware and the like, and may also work in conjunction with other hardware, to accomplish programmed tasks.

As used herein, the term "short-range wireless communications protocol" refers to protocols for wireless communication at distances of up to about 10 meters, though one or more on the following list may go further in practice. In addition, new or improved forms of short-range wireless communications protocols may extend the range. Examples include Bluetooth, Bluetooth Low Energy, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

As used herein, “care services” refer to services for the adults that encompass a range of non-medical and personal support designed to help adults of advanced age (e.g., at least 65 years old) and/or infirmed adults maintain their independence, health, and quality of life. These services range from in-home companion care to assisted living. For example, as individuals age, complex health conditions and mobility issues can make it difficult to manage everyday activities independently. Care services are crucial because they promote independence, manage health and safety, nurture well-being, and/or provide family support.

Elder care can be tailored to an individual's unique physical, mental, and emotional health needs. Common types of care can include personal care, home health care, companion and homemaker services, and residential care. Personal care can involve assistance with daily tasks such as bathing, dressing, grooming, and mobility. Home health care can refer to skilled services provided at home, including nursing care, medication management, physical therapy, and chronic disease management. Companion and homemaker services may offer support with tasks like grocery shopping, meal preparation, and light housekeeping, along with social interaction to prevent isolation. Residential care can include facilities like assisted living communities or nursing homes that provide 24/7 supervision, security, and daily support.

In the past, communication devices for care services have typically been limited to a single, basic "call button," where the care recipient (i.e., the person requesting the care service task) presses a single button to send a notification to the caregiver (i.e., the person selected by the recipient to perform a care service task). However, such notifications typically do not provide information regarding the nature of the alert or the associated level of urgency of the status message and fail to limit unnecessary communications between the caregiver and the care recipient.

The instant disclosure seeks to provide cellular enabled communications device configured to provide a more nuanced messaging system that allows care recipients to tailor the notification to a specific type of care service task and thereby provide caregivers enhanced understanding of the care recipient's needs prior to responding. For example, requests for self-care tasks (e.g., hydration assistance), personal care tasks (e.g., toileting assistance), and comfort care tasks (e.g., physical comfort assistance and physical pain management assistance) carry varying degrees of urgency and stress for caregivers, unlike a traditional "help" button, which always conveys the same urgency. The present disclosure seeks to provide a notification system and device that minimizes communication between the caregiver and care recipient. The instant disclosure seeks to provide notification devices that provide care recipients minimal communications solutions to request assistance with one or more self-care tasks.

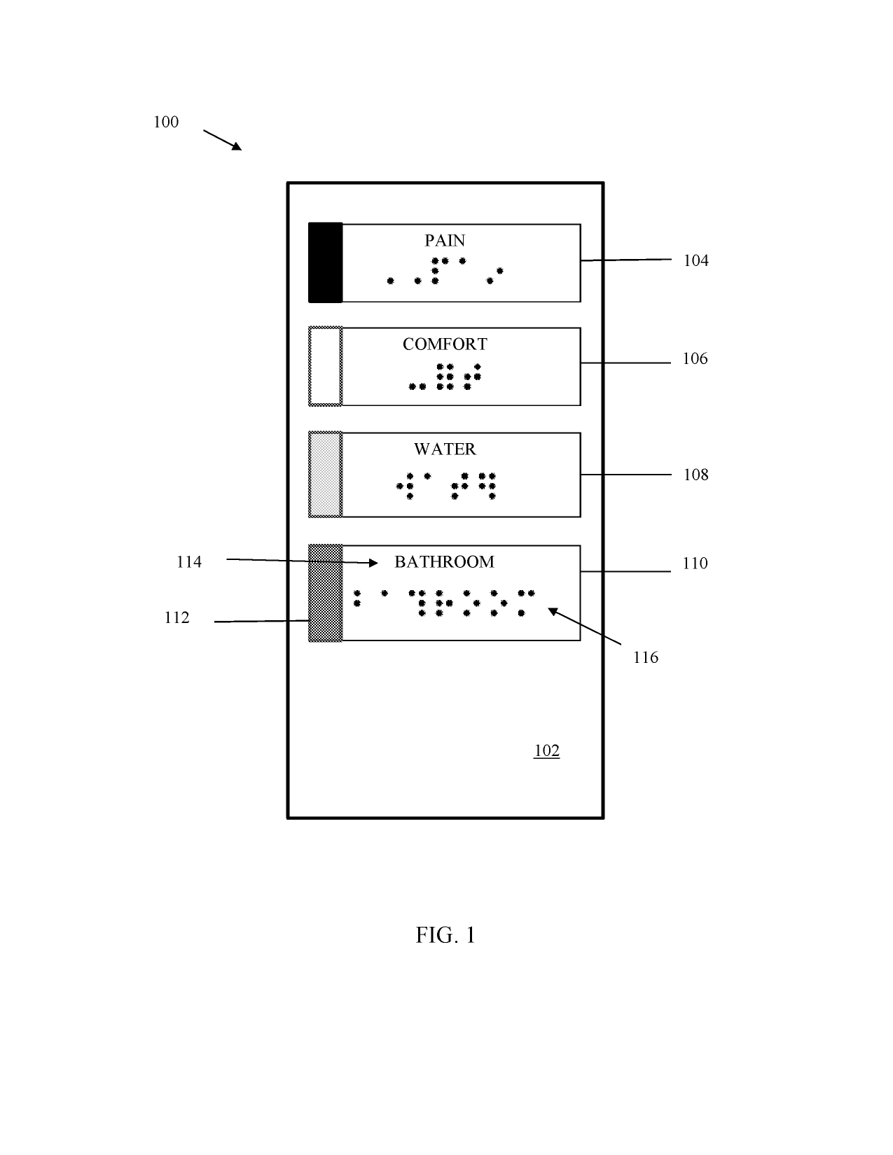

Referring now to the drawing, FIG. 1 depicts one example of a simplified communications device 100, in accordance with one or more aspects of the instant disclosure. The simplified communications device 100 may include, for example, a housing 102 and four keys 104, 106, 108 and 110. Although the example of FIG. 1 includes four keys, it will be understood that there could be as few as two keys or more than four keys. Each key corresponds to a different status message of a care recipient. The status messages in this example include, "pain," "comfort," "water" and "bathroom," each message corresponding to a key 104, 106, 108 and 110, respectively.

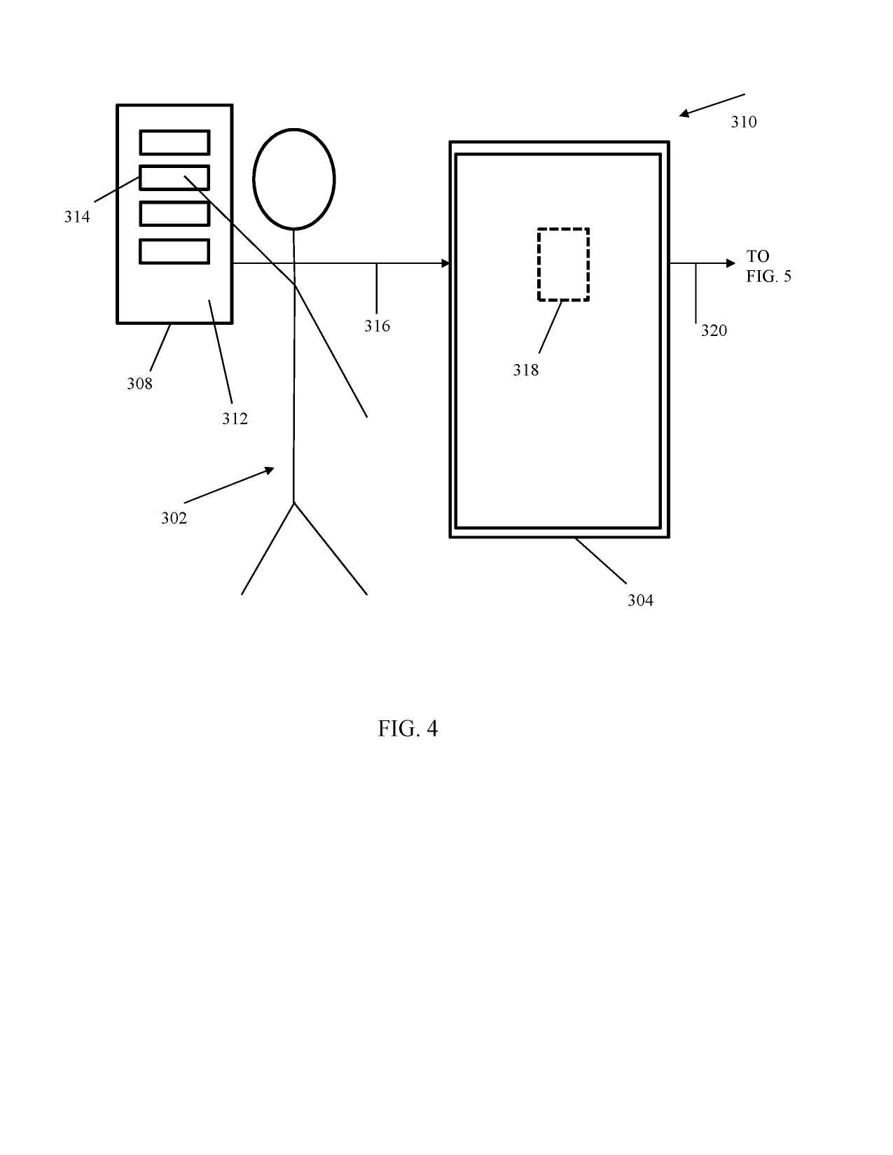

In one embodiment, each key includes one or more forms of a descriptor thereon or in close proximity thereto, the descriptor indicating the corresponding message. For example, key 110 includes a color or grayscale 112 different from colors or grayscale of the other keys. As another example of a message descriptor, text 114 may be included. As another example, the message descriptor(s) may include raised Braille characters 116. In one example, the keys may include one or more touch screens (e.g., a single touch screen including areas thereof for each of the keys, or four separate, smaller touch screens). In one example, the one or more touch screens may include haptic feedback or another form of feedback. In another example, the keys may include physical keys, preferably physical keys that "click" to provide feedback. Preferably, the keys are spaced apart for ease of use and to help ensure two or more adjacent keys are not pressed together. In one example, there are four keys 104, 106, 108 and 110 spaced apart from each other on the simplified communications device 308 in a vertical column, as in FIG. 4.

In some cases, the person receiving care will be mobile and will carry or attach a lanyard to the simplified communications device. In other cases, the person receiving care may not be mobile and may be in a bed. In that case, the person receiving care will have the simplified communications device on the bed or a nightstand or tray. Alternatively, the simplified communications device could be attached to something stationary in close proximity to the person receiving care, preferably a removable attachment, to, for example, a side rail of the bed.

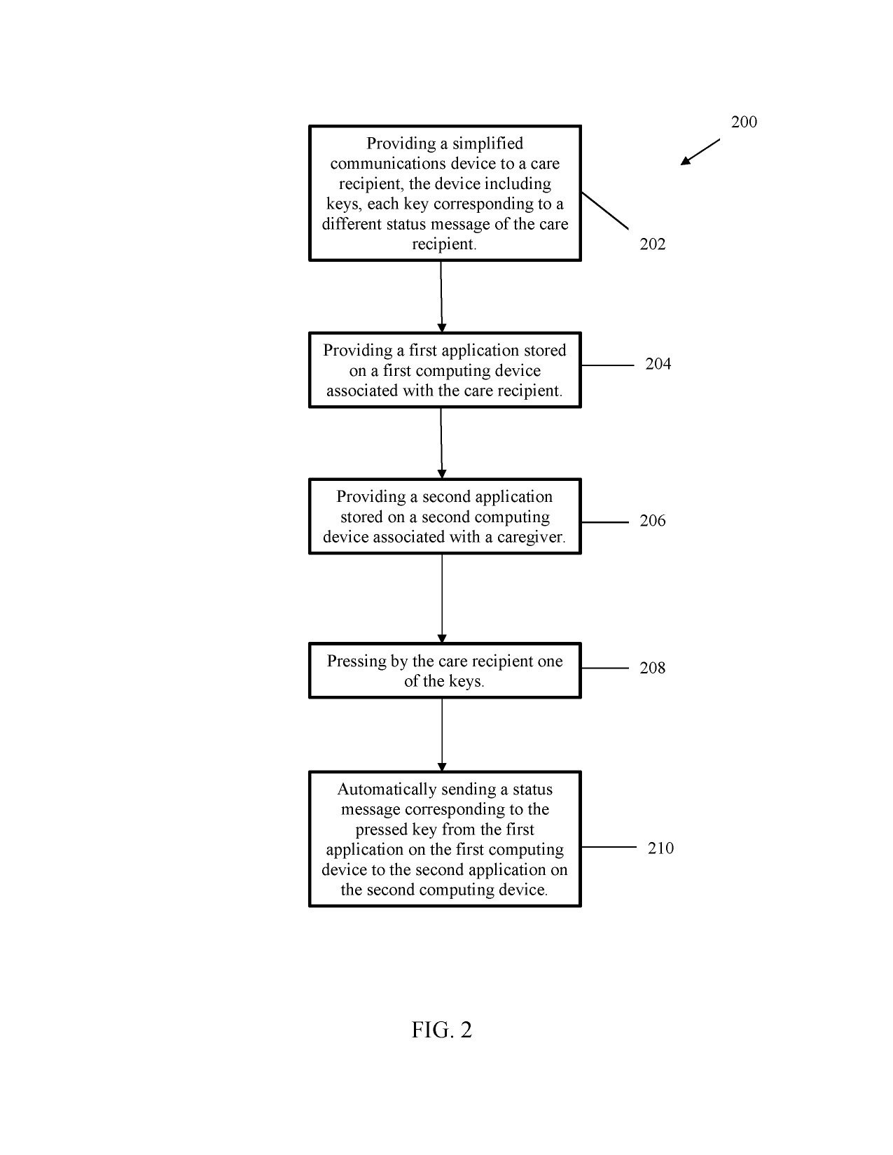

FIG. 2 is a flow diagram 200 for one example of a method of communicating a status of a care recipient using the simplified communications device of FIG. 1, in accordance with one or more aspects of the instant disclosure. The method may include, for example, providing a simplified communications device to a care recipient, the simplified communications device including at least two keys, each key corresponding to a different status message of the care recipient, step 202. The method may also include, for example, providing a first application stored on a first computing device associated with the care recipient, step 204. The method may further include, for example, providing a second application stored in a memory of a second computing device associated with a caregiver, step 206. The method may also include, for example, pressing by the care recipient one of the keys of the simplified communications device, step 208. The method may further include, for example, automatically sending a status message corresponding to the pressed key from the first application on the first computing device to the second application on the second computing device, step 210.

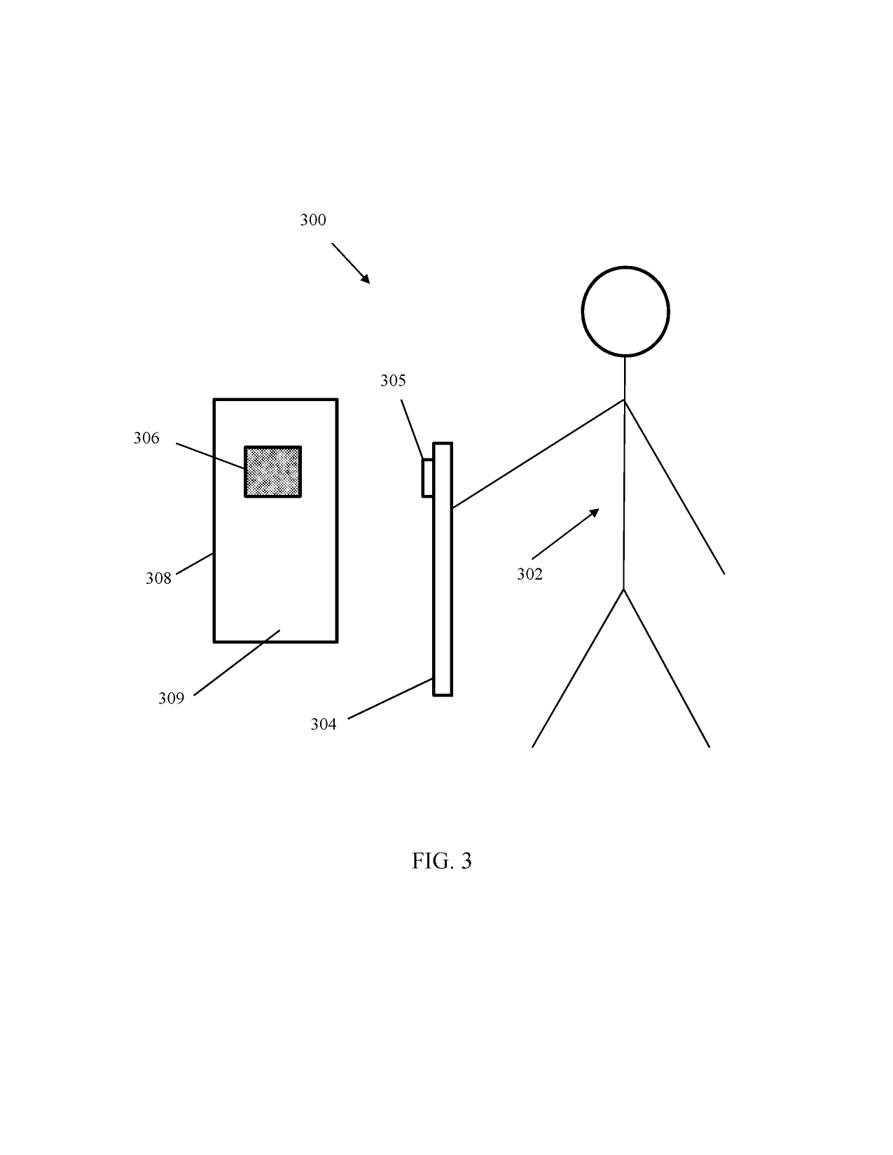

FIGS. 3-5 depict examples of various stages of performing the method of the instant disclosure. FIG. 3 depicts, not to scale for ease of viewing, one example of requesting an application 300 for a simplified communications device 308 by a care recipient 302, in accordance with one or more aspects of the instant disclosure. For example, the requesting may include requesting an application for the simplified communications device from a manufacturer of the simplified communications device or a business entity associated with providing a status message service. The care recipient 302 may, for example, use a first computing device 304 associated with the care recipient 302 to perform steps of the method. The first computing device 304 may be owned, rented, leased by or lent to the care recipient 302. For example, the first computing device 304 may be part of the subscription service or simply owned by the care recipient 302. In this regard, the care recipient 302 may use an imaging sensor 305 on the first computing device 304 (e.g., via a photo application or dedicated QR code reader application) to scan, for example, a "QR" code 306 on a simplified communications device 308 of the instant disclosure. As one skilled in the art will know, a QR code (short for "quick-response code") is a type of two-dimensional matrix barcode. In one example, the QR code 306 may be placed, for example, on a back side 309 of the simplified communications device 308, while the keys may be placed, for example, on a front side thereof (312, FIG. 4). Other possible methods to request an application for the simplified communications device 308 include, for example, calling a call center associated with the business entity, having a video chat with a customer service representative, manually requesting an application for the simplified communications device 308 online, e.g., on a web site associated with the business entity or via email or text messaging.

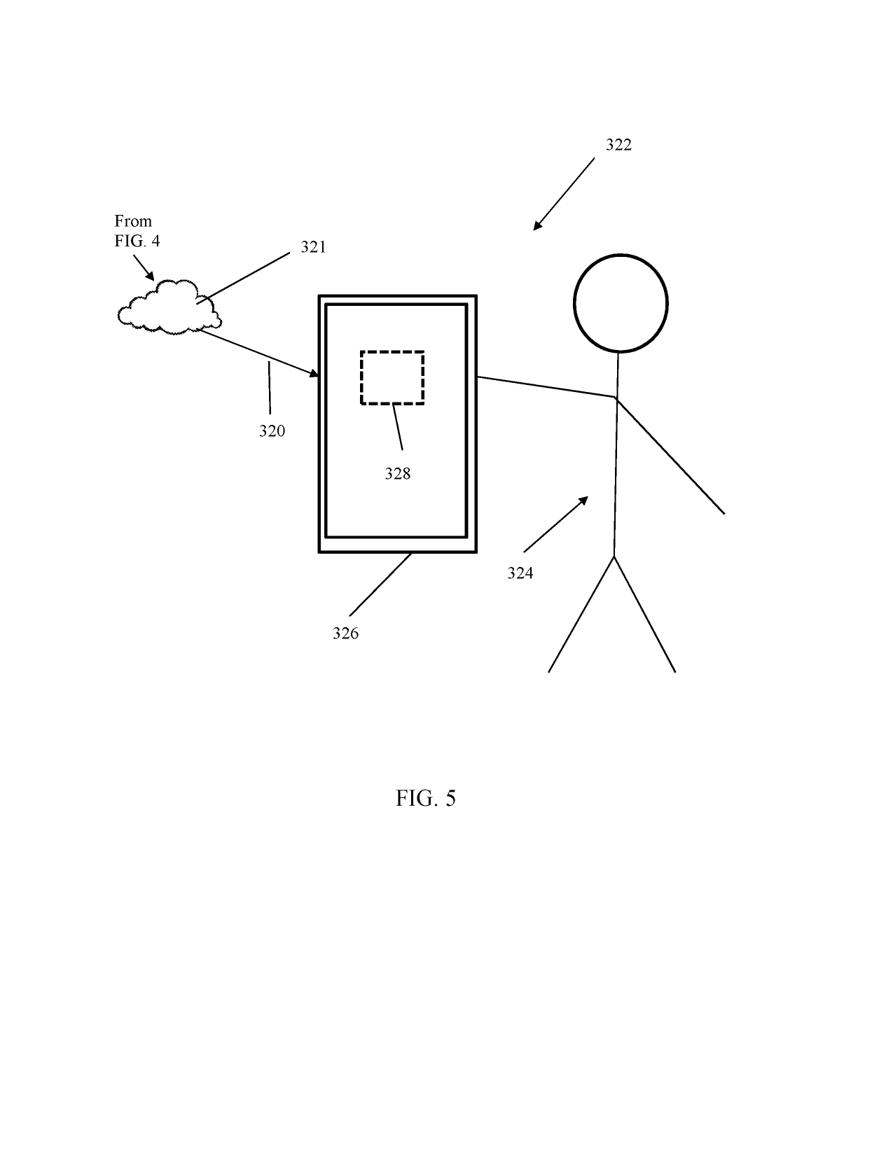

FIG. 4 depicts, not to scale for ease of viewing, one example 310 of the care recipient 302 of FIG. 3 pressing one of the keys (e.g., key 314) on the simplified communications device 308, in accordance with one or more aspects of the instant disclosure. Based on one of the keys being pressed, a signal 316 is sent from the simplified communications device 308 to an application 318 executing on the first computing device 304. Based on receipt of the signal 316, for example, over a network (321, FIG. 5) (e.g., the Internet, a private network, personal area network or a cellular network) from the simplified communications device 308, a status message 320 of the care recipient corresponding to the key 314 pressed may be, for example, automatically sent from the application 318 executing on the first computing device 304 to a second application (328, FIG. 5) executing on the second computing device (326, FIG. 5).

FIG. 5 depicts, not to scale for ease of viewing, one example 322 of a caregiver 324 receiving on a second computing device 326 associated with the caregiver, a status of the care recipient 302 in response to the care recipient 302 pressing the key 314 of FIG. 4, in accordance with one or more aspects of the instant disclosure. The status message 320 is then received by the second application 328 executing on the second computing device 326. At that point, the caregiver may act on the status message by, for example, physically checking on the care recipient, calling the care recipient, emailing or texting the care recipient. As another example, the caregiver may forward the status message to another family member or colleague, or to a team of caregivers for the care recipient.

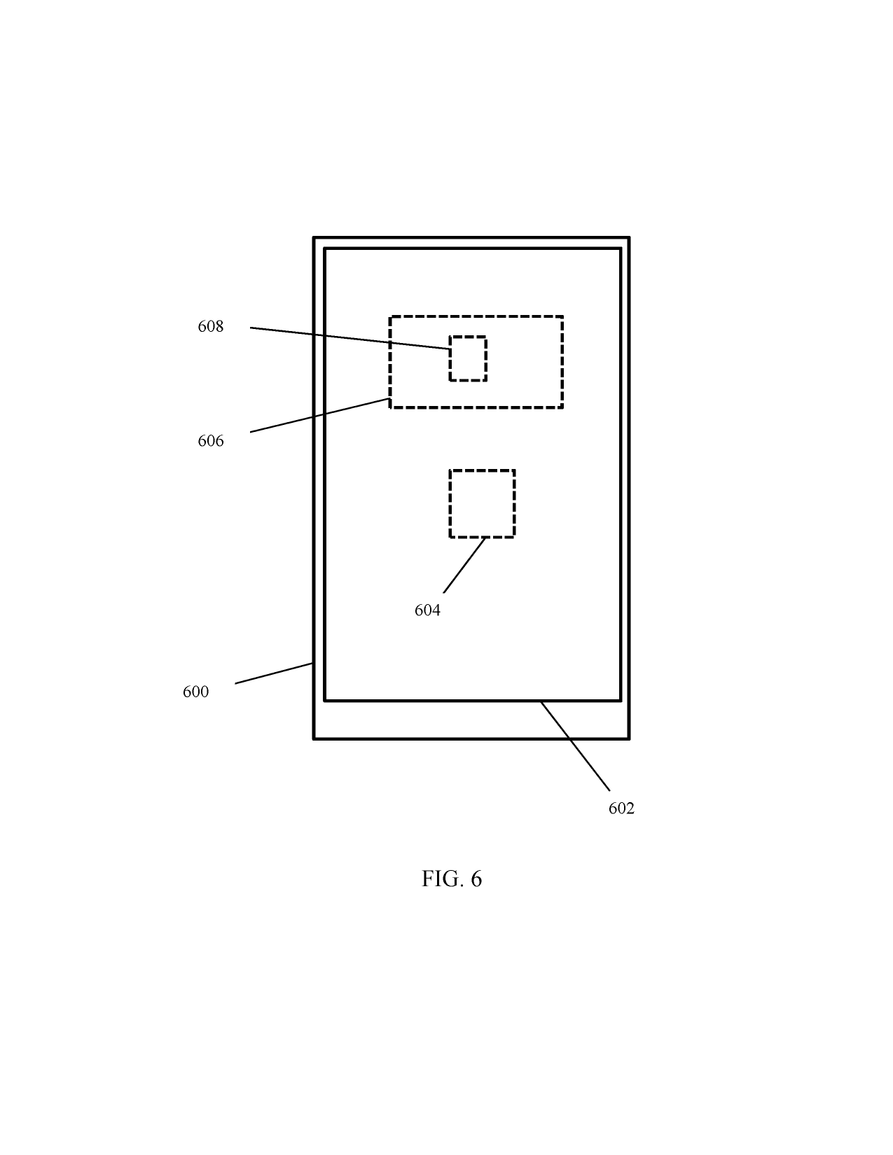

FIG. 6 depicts one example of a computing device 600, in accordance with one or more aspects of the instant disclosure. The computing device includes, for example, a screen 602 that may or may not be bendable or foldable, one or more processors 604 and memory 606 in which is stored one or more applications 608, for example, an operating system, photo application or other general or special purpose applications. Examples of memory include, but are not limited to, random access memory (RAM), dynamic random access memory (DRAM), solid state memory, read only memory (ROM), programmable ROM, erasable programmable ROM, electrically erasable programmable ROM, flash memory, cache memory, double data rate synchronous DRAM (DDR), volatile memory, double data rate 4 synchronous dynamic RAM (DDR4 SDRAM), double data rate 2 synchronous dynamic RAM (DDR2), double data rate 3 synchronous dynamic RAM (DDR3), static RAM and erasable programmable ROM (EPROM).

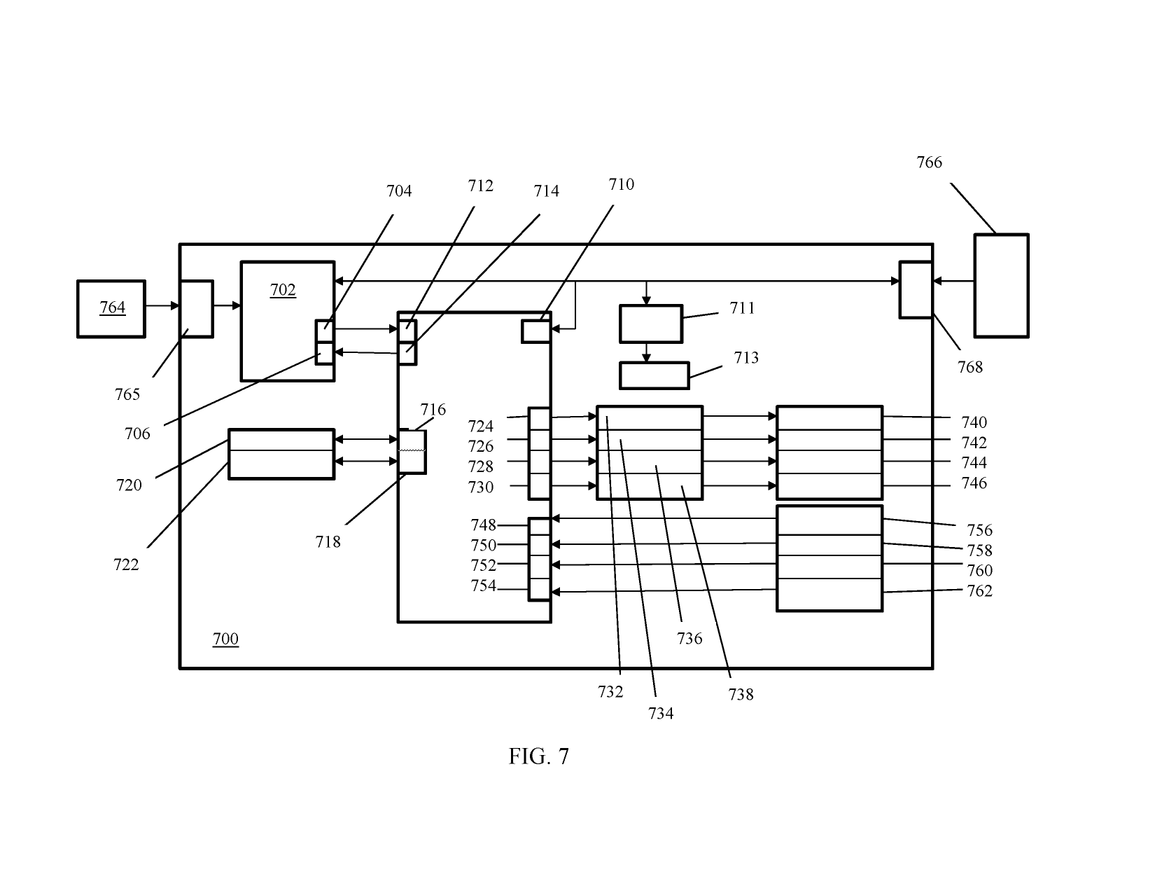

FIG. 7 depicts one example of a printed circuit board assembly 700 ("PCBA") for the simplified communications device 100 of FIG. 1, in accordance with one or more aspects of the instant disclosure. The PCBA may include, for example, a wireless power battery charger 702, having an output 704 (e.g., a Schmitt Trigger or "ST") and an input 706 (e.g., an Energy Note or "EN"). One example of a commercially available wireless power battery charger is model BQ5105xB from Mouser Electronics, Inc. in Mansfield, Texas. Also included in the PCBA 700 is a multiprotocol module 708; in this case, it has Bluetooth and Wi-Fi protocols. One example of a commercially available multiprotocol module is ESP-C6-MINI-1 from Espressif Systems located in Shanghai, China. Multiprotocol module 708 may include, for example, an analog-to-digital converter 710, input 712 and output 714. The analog to digital converter 710 may have a bidirectional connection to a buck regulator 711 and VCC 713 (about 3.6 volts). Multiprotocol module 708 may further include, for example, input/output 716 and 718, each with bidirectional capability, for JTAG 720 and UART 722, respectively. As one skilled in the art will know, "JTAG" stands for joint test action group. JTAG is an integrated method for testing interconnects on printed circuit boards (PCBs) that are implemented at the integrated circuit (IC) level and "UART" stands for universal asynchronous receiver / transmitter and defines a protocol, or set of rules, for exchanging serial data between two devices. Also included in multiprotocol module 708 are four outputs 724, 726, 728 and 730, each driving an LED driver 732, 734, 736 and 738, respectively. The LED drivers 732, 734, 736 and 738 control LEDs 740, 742, 744 and 746, respectively. The multiprotocol module 708 may include, for example, inputs 748, 750, 752 and 754 for input from keys 756, 758, 760 and 762, respectively, electrically connected to keys 104, 106, 108 and 110 in the simplified communications device 100 of FIG. 1. In addition, to facilitate wireless charging is a charging coil 764 (e.g., 25 watts) to charge through connector 765 and battery 766 (e.g., a lithium polymer battery of 2000 mAh capacity) via connector 768.

In a first aspect, the invention includes a simplified communications device. In one embodiment, the simplified communications device may include, for example, a housing and keys coupled to the housing, each key corresponding to a different status message of a care recipient. The simplified communication device may further include, for example, a first memory situated within the housing and storing a first application and may also include, for example, a processor situated within the housing, the processor configured to, in conjunction with the first application, cause the first application on the first computing device to send a status message corresponding to one of the keys, the simplified communications device being capable of a wireless connection to the first computing device associated with the care recipient; and based on one of the plurality of keys being pressed, the status message is automatically sent from the first application executing on the first computing device to a second application executing on a second computing device associated with a caregiver.

In another example, the wireless connection of the simplified communications device of the first aspect may use, for example, a short-range wireless communications protocol. As examples, the short-range wireless communications protocol may include, for example, one of Bluetooth, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

In still another example, the first application and the second application of the first and second computing devices, respectively, may be, for example, a same application.

In yet another example, the second application may be stored in a second memory associated with the second computing device. In one example, each of the first memory and the second memory may include one of an integrated memory and a removable memory (e.g., a flash memory device).

In another example, the simplified communications device of the first aspect may further include one or more descriptors indicating status messages associated with the keys. For example, the descriptor may take the form of at least one of a different color or grayscale for each of the plurality of keys, descriptive Braille on or adjacent to each of the plurality of keys and descriptive text on or adjacent to each of the keys.

In still another example, the keys may include, for example, physical keys.

In yet another example, the keys of the simplified communications device of the first aspect may include, for example, one or more capacitive touch screens with or without haptic or other feedback, such as, for example, an LED light, a blinking LED light, various colors of LED lighting or blinking LED lighting of various colors.

In a second aspect, the invention includes a method of communicating a status of a care recipient. The method may include, for example, providing a simplified communications device and a first application to the care recipient, the first application being made available, for example, by downloading the same. The simplified communications device may include, for example, a housing and keys coupled to the housing, each key corresponding to a different status message of a care recipient. The simplified communications device may further include, for example, a first memory situated within the housing and storing the first application and may also include, for example, a processor situated within the housing, the processor configured to, in conjunction with the first application, cause the first application on the first computing device to send a status message corresponding to one of the keys, the simplified communications device being capable of a wireless connection to the first computing device associated with the care recipient. The method may further include, for example, providing a second application, for example, by making it available for download, stored in a second memory associated with a second computing device associated with a caregiver and based on one of the plurality of keys being pressed, automatically sending a status message corresponding to the one of the plurality of keys from the first application executing on the first computing device to the second application executing on the second computing device.

In one example, the keys of the simplified communications device in the method of the second aspect may include two or more keys.

In another example, the wireless connection of the simplified communications device in the method of the second aspect may use, for example, a short-range wireless communications protocol. In examples, the short-range wireless communications protocol may include, for example, one of Bluetooth, Wi-Fi, a combination of low-power connectivity and a high-speed connection, near-field communication (NFC), radio frequency identification (RFID), low-power wide area network (LPWAN), ultra-wideband (UWB) and low-rate wireless personal area network (LR-WPAN).

In still another example, the first application and the second application of the simplified communications device may be, for example, a same application. In another example, where the same application is provided to both the care recipient and the caregiver, the application may look and/or function differently for each person in accordance with their roles.

In yet another example, in the method of the second aspect, the second application may be stored, for example, in a second memory associated with the second computing device. In one example, each of the first memory and the second memory may include one of an integrated memory and a removable memory (e.g., a flash memory device).

In another example, the simplified communications device of the first aspect may further include at least one of: a different color or grayscale for each of the keys; descriptive Braille on or adjacent to each of the plurality of keys and descriptive text on or adjacent to each of the keys.

In yet another example, the keys of the simplified communications device of the method of the second aspect may include, for example, one or more capacitive touch screens with or without haptic feedback or other feedback, such as, for example, one or more different sounds, an LED light, blinking LED light, various colors of LED lighting or blinking LED lighting of various colors.

In another example, providing the simplified communications device may include, for example, requesting an application for the simplified communications device. Further, in another example, providing the first application and the second application may each include making the respective application available for download.

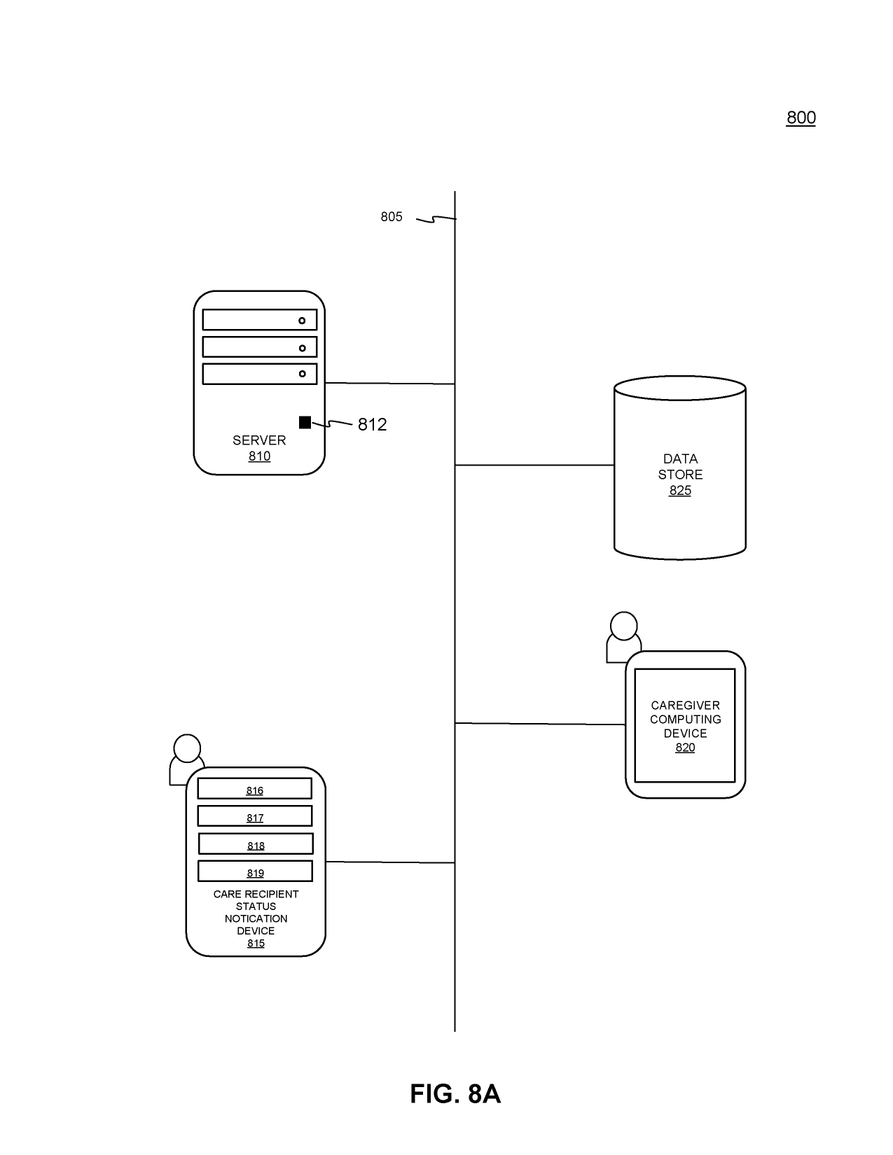

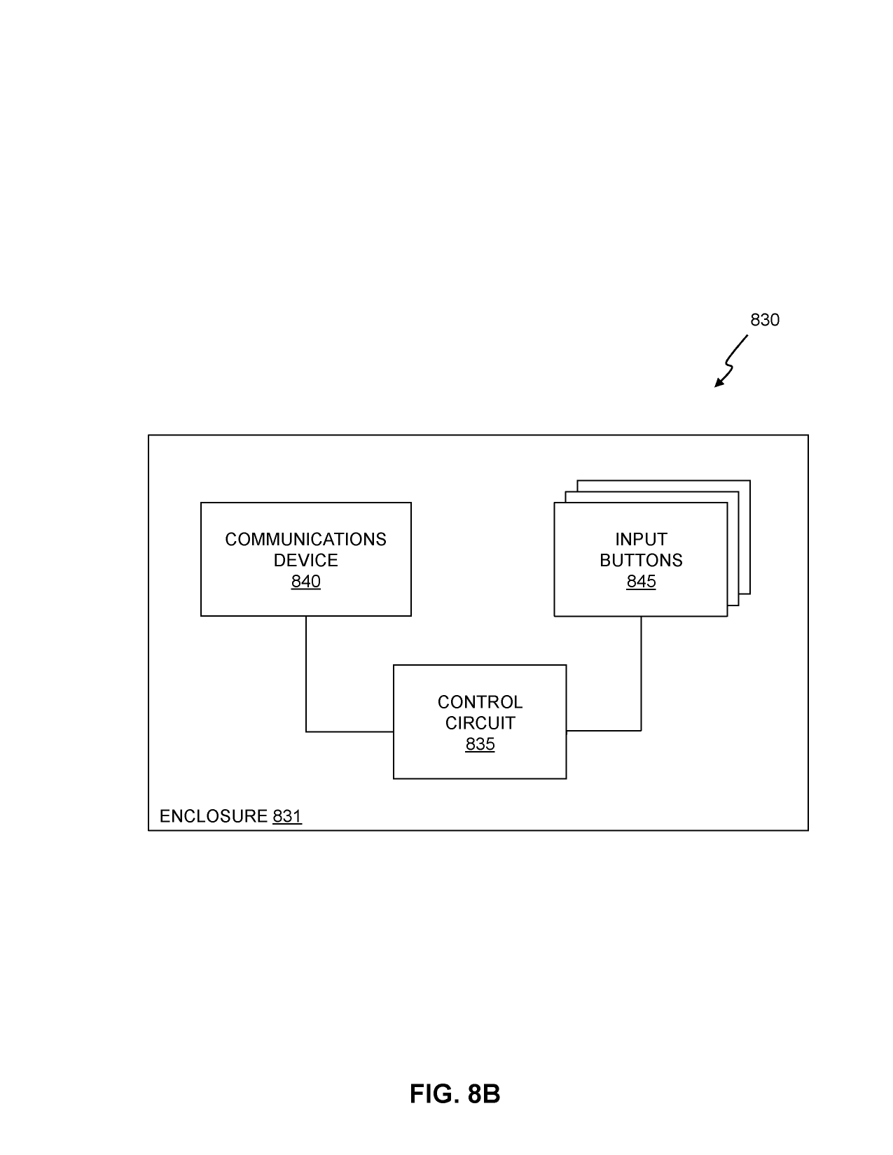

FIG. 8A is a block diagram illustrating a status message data processing environment, generally 800, in accordance with some embodiments of the instant disclosure. Environment 800 includes server 810, care recipient status notification (CRSN) device 815, and caregiver computing device 820, all interconnected over network 805. Network 805 can be, for example, a local area network (LAN), a wide area network (WAN) such as the Internet, or a combination of the two, and can include wired, wireless, or fiber optic connections. In general, network 805 can be any combination of connections and protocols that will support communications between server 810, CRSN device 815, and caregiver computing device 820 using one or more cellular network protocols known in the art.

In preferred embodiments, network 805 includes a telecommunications network in which communication links between end nodes and the network infrastructure are established wirelessly. The network is geographically partitioned into a plurality of coverage areas, or “cells,” each of which is served by at least one fixed-location transceiver, such as a base station. Accordingly, network 805 may be characterized as a cellular network. Each base station is configured to provide wireless coverage within its corresponding cell, thereby enabling the transmission and reception of voice, data, and other content via radio frequency (RF) signals.

The effective coverage area of a given cell is determined by various factors, including, but not limited to, transceiver power, local terrain and environmental conditions, antenna characteristics, and the frequency band utilized. To mitigate interference and maintain quality of service, adjacent cells are typically assigned different frequency resources or otherwise employ frequency reuse schemes, such that co-channel interference is minimized while overall spectral efficiency is preserved. This arrangement enables reliable communication within each cell while supporting scalable network capacity across the broader service area.

In various embodiments of the present invention, server 810 and caregiver computing device 820 may be a laptop computer, tablet computer, netbook computer, personal computer (PC), a desktop computer, a personal digital assistant (PDA), a smart phone, or any programmable electronic device capable of communicating with each other and/or CRSN device 815 via network 805. In preferred embodiments, caregiver computing device 820 is a mobile computing device configured to communicate via one or more cellular network protocols. Server 810 preferably includes caregiver program 812. Alternatively, caregiver program 812 can be stored on data store 825. Caregiver program 812 is software that facilitates communication between CRSN device 815 and caregiver computing device 820 as disclosed in the instant disclosure.

CRSN device 815 is an apparatus that conveys one or more (e.g., up to four) care service task notifications selected by the care recipient to caregiver computing device 820. In certain embodiments, the only input devices included in CRSN device 815 include input buttons 816, 817, 818, and/or 819. As used herein, a “button” refers to a graphical or physical control element used to trigger the transmission of signals to server 810 to thereby cause server 810 to transmit the status message to the computing device when pressed.

As depicts in FIG. 8B, CRSN device 815 can include enclosure 831 that includes a plurality of input buttons 845 (e.g., input buttons 816, 817, 818, and/or 819), communications device 840, each communicatively coupled to control circuit 835. CRSN device 815 can be registered on caregiver program 812 to be exclusively linked to caregiver computing device 820. For example, CRSN device 815 can include a unique QR code (or other two-dimensional or three-dimensional matrix barcode) and serial number linking the device to its registered caregiver(s). In certain embodiments, CRSN device 815 includes a Subscriber Identity Module (SIM) card reader/dock. Similarly, caregiver computing device 820 can be registered on caregiver program 812 to be exclusively linked to CRSN device 815. Such registration ensures that notifications initiated by CRSN device 815 are exclusively sent to preauthorized caregiver computing devices. Each input buttons 845 can be associated with an unique input signal. Each input signal is preferably associated with a unique status message. Control circuit 835 can be configured to cause server 810 to transmit a status message to caregiver computing device 820 when the input signal is received by server 810.

In preferred embodiments, status messages are conveyed as text messages (e.g., SMS text message). Communications device 840 is configured to communicate with server 810 using one or more cellular network communications protocols (e.g., 4G Long-Term Evolution (LTE), LTE-Advanced, 5G New Radio, cellular IoT protocols, Short Message Service (SMS), and/or other cellular network communications protocols.

For example, each input button 845 sends a signal to control circuit 835 that translates to an operational command to cause server 810 to transmit the status message to caregiver computing device 820 when pressed. Specifically, in response to receiving the signal, control circuit 835 is configured to use communication device 840 to transmit the operational command to server 810 to thereby cause server 810 to transmit the status message to caregiver computing device 820.

Care recipient uses CRSN device 815 to convey one or more status messages to the registered caregiver via caregiver computing device 820. Applicable status messages can be associated with requests for assistance with a self-care task (e.g., hydration assistance), a personal care task (e.g., toileting assistance), and/or a comfort care task (e.g., physical comfort assistance and/or pain management assistance). In other embodiments, caregiver computing device 820 is mobile device used by (i.e., associated with) a non-medical person selected by the care recipient to perform one or more non-medical tasks (i.e., the care service tasks) for the care recipient. Each non-medical task is directly associated with one of the status messages.

For example, hydration assistance requests typically result in a non-medical task that involves the caregiver addressing the care recipient’s hydration concerns (e.g., directly or indirectly providing a hydration solution); toileting assistance requests typically result in a non-medical task that involves the caregiver addressing the recipient’s toileting concerns (e.g., safely accessing a bathroom, removing clothing, using the toilet, cleaning oneself, redressing, and/or washing hands); physical comfort assistance requests typically result in non-medical tasks that involve the caregiver addressing the recipient’s physical comfort concerns (e.g., adjusting positioning); and pain management assistance requests typically result in non-medical tasks involving the caregiver addressing the recipient’s pain management concerns (e.g., assistance with medication administration). Alternatively, or in addition, CRSN device 815 receives an audible input from the care recipient that triggers the conveyance of the status message. In other embodiments, CRSN device 815 receives the trigger event from a smart assistance. For example, the care recipient may inform the smart assistant of their service need and in response, the smart assistant sends the trigger event to CRSN device 815 to convey the status message.

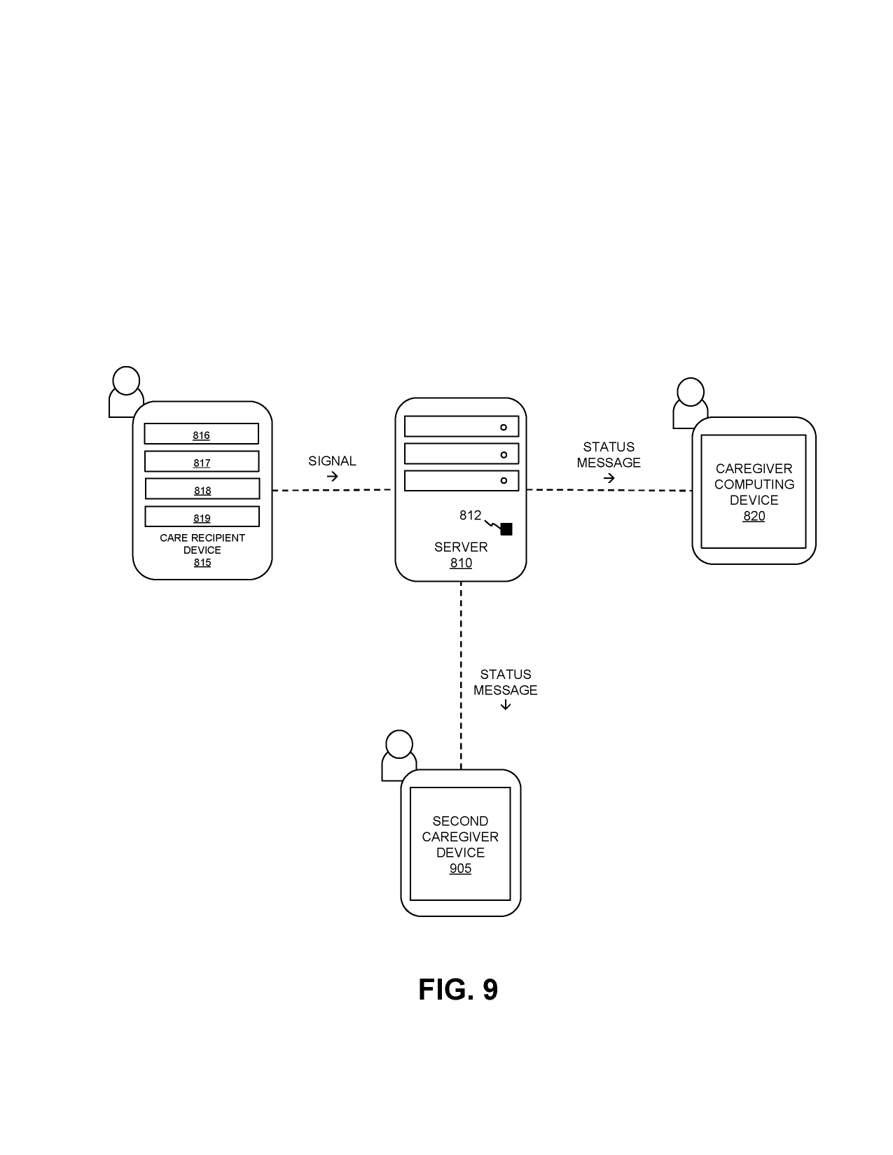

FIG. 9 is a block diagram illustrating the information flow of the status message data processing environment 800 involving a plurality of registered caregiver computing devices, in accordance with other embodiments of the instant disclosure In some embodiments, care recipients can use caregiver program 812 to register their CRSN device with a plurality of caregiver computing devices 820 and thereby allow server 810 to transmit their status messages to a second computing device (e.g., second caregiver computing device 905 registered to a backup/secondary caregiver) when the confirmation signal from the primary caregiver device is not received within a predetermined time period. In preferred embodiments, the second computing device is a mobile device.

When multiple caregiver computing devices 905 are registered with caregiver program 812 to a care recipient, each caregiver can schedule (provide the date and time) their availability (e.g., Device A is available daily from 9:00 AM to 5:00 PM, and Device B is available from 5:00 PM to 12:00 AM), which allows caregiver program 812 to determine the primary and secondary devices. In certain embodiments, caregivers can use caregiver computing devices to send a caregiver confirmation signal to server 810 when the non-medical task has been performed or confirming that the status message was received. Caregiver program 812 can prevent duplicate status messages from being transmitted to caregiver computing device 905 (or other caregiver devices) when a button is pressed multiple times within a predetermined time span.

In certain embodiments, multiple caregiver computing devices (e.g., caregiver computing devices 820 and 905) are registered with server 810 to receive the care recipient’s status messages in a tiered manner triggered by a delay in server 810 receiving the caregiver confirmation signal. For example, control circuit 835 is configured to receive a confirmation signal from server 810 when server 810 receives the caregiver confirmation signal from the caregiver computing device when the non-medical task has been performed. Caregiver program 812 can prompt caregiver computing device 820 (or other caregiver device) to confirm whether the request has been addressed, helping close the loop and potentially update device indicators.

For example, in response to receiving the caregiver confirmation signal from caregiver computing device 820, server 810 transmits a care recipient confirmation signal to CRSN device 815 to thereby cause the CRSN device 815 to convey the confirmation using a non-display output device. Applicable non-display output devices include, but are not limited to, haptic feedback devices, speakers, light sources (e.g., LEDs), and/or other non-display output devices).

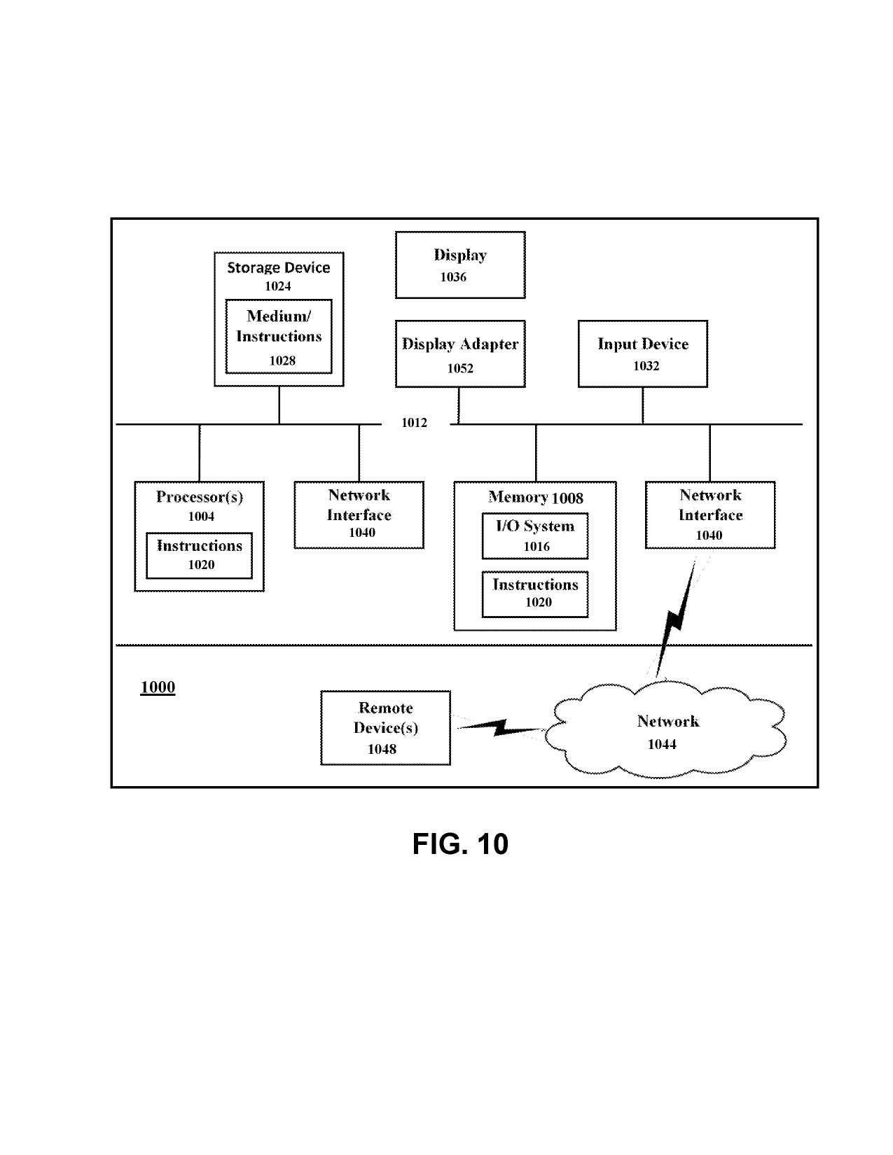

FIG. 10 depicts a block diagram of components of server 810 executing caregiver program 812, in accordance with certain embodiments of the present disclosure. FIG. 10 shows an example embodiment of a computing system for implementing embodiments of the disclosure. Computer system 1000 includes a processor 1004 and a memory 1008 that communicate with each other, and with other components, via a bus 1012. Bus 1012 can include any of several types of bus structures including, but not limited to, a memory bus, a memory controller, a peripheral bus, a local bus, and any combinations thereof, using any of a variety of bus architectures.

Memory 1008 can include various components (e.g., machine-readable media) including, but not limited to, a random-access memory component, a read-only component, and any combinations thereof. In one example, a basic input/output system 1016 (BIOS), including basic routines that help to transfer information between elements within computer system 1000, such as during start-up, can be stored in memory 1008. Memory 1008 can also include (e.g., stored on one or more machine-readable media) instructions (e.g., software) 1020 embodying any one or more of the aspects and/or methodologies of the present disclosure. In another example, memory 1008 can further include any number of program modules including, but not limited to, an operating system, one or more application programs, other program modules, program data, and any combinations thereof.

Computer system 1000 can also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 can be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) can be removably interfaced with computer system 1000 (e.g., via an external port connector (not shown)). Particularly, storage device 1024 and an associated machine-readable medium 1028 can provide nonvolatile and/or volatile storage of machine-readable instructions, data structures, program modules, and/or other data for computer system 1000. In one example, instructions 1020 can reside, completely or partially, within machine-readable medium 1028. In another example, instructions 1020 can reside, completely or partially, within processor 1004.

Computer system 1000 can also include an input device 1032. In one example, a user of computer system 1000 can enter commands and/or other information into computer system 1000 via input device 1032. Examples of an input device 1032 includes, but are not limited to, an alphanumeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 can be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 can include a touch screen interface that can be a part of or separate from display device 1036, discussed further below. Input device 1032 can be utilized as a user selection device for selecting one or more graphical representations in a graphical interface as described above.

A user can also input commands and/or other information to computer system 1000 via storage device 1024 (e.g., a removable disk drive, a flash drive, etc.) and/or network interface device 1040. A network interface device, such as network interface device 1040, can be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network, such as network 1044, can employ a wired and/or a wireless mode of communication. In general, any network topology can be used. Information (e.g., data, instructions 1020, etc.) can be communicated to and/or from computer system 1000 via network interface device 1040.

Computer system 1000 can further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Video display adapter 1052 and display device 1036 can be utilized in combination with processor 1004 to provide graphical representations of aspects of the present disclosure. In addition to a display device, computer system 1000 can include one or more other peripheral output devices including, but not limited to, an audio speaker, a printer, and any combinations thereof. Such peripheral output devices can be connected to bus 1012 via a peripheral interface 1056. Examples of a peripheral interface include, but are not limited to, a serial port, a USB connection, a FIREWIRE connection, a parallel connection, and any combinations thereof.

It is understood that the present subject matter may be embodied in many different forms and should not be construed as being limited to the embodiments set forth herein. Rather, these embodiments are provided so that this subject matter will be thorough and complete and will convey the disclosure to those skilled in the art. Indeed, the subject matter is intended to cover alternatives, modifications, and equivalents of these embodiments, which are included within the scope and spirit of the subject matter as defined by the appended claims and their equivalents. Furthermore, in the detailed description of the present subject matter, numerous specific details are set forth in order to provide a thorough understanding of the present subject matter. However, it will be clear to those of ordinary skill in the art that the present subject matter may be practiced without such specific details.

Aspects of the present disclosure are described herein with reference to flowchart illustrations and/or block diagrams of methods, apparatuses (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

The non-transitory computer-readable media includes all types of computer-readable media, including magnetic storage media, optical storage media, and solid-state storage media and specifically excludes signals. It should be understood that the software can be installed in and sold with the device. Alternatively, the software can be obtained and loaded into the device, including obtaining the software via a disc medium or from any manner of network or distribution system, including, for example, from a server owned by the software creator or from a server not owned but used by the software creator. The software can be stored on a server for distribution over the Internet, for example.

Computer-readable storage media (medium) exclude (excludes) propagated signals per se, can be accessed by a computer and/or processor(s), and include volatile and non-volatile internal and/or external media that is removable and/or non-removable. For the computer, the various types of storage media accommodate the storage of data in any suitable digital format. It should be appreciated by those skilled in the art that other types of computer readable medium can be employed such as zip drives, solid state drives, magnetic tape, flash memory cards, flash drives, cartridges, and the like, for storing computer executable instructions for performing the novel methods (acts) of the disclosed architecture.

The terminology used herein is for the purpose of describing particular aspects only and is not intended to be limiting of the disclosure. As used herein, the singular forms "a," "an" and "the" are intended to include the plural forms as well, unless the context clearly indicates otherwise. It will be further understood that the terms "comprises" and/or "comprising," when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

For purposes of this document, each process associated with the disclosed technology may be performed continuously and by one or more computing devices. Each step in a process may be performed by the same or different computing devices as those used in other steps, and each step need not necessarily be performed by a single computing device.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A care recipient status notification (CRSN) device, comprising:
an enclosure that comprises:
a plurality of input buttons;
a communications device;
a control circuit communicatively coupled to each of said input buttons and the communications device;
wherein
the CRSN device is configured to be registered to and used by a care recipient;
each of said input buttons are associated with an input signal that is unique to that input button;
each input signal is associated with a status message that is unique to the input signal;
the control circuit configured to cause a server to transmit the status message to a computing device when the input signal is received, the status message is a text message; and
the server configured to transmit data via a cellular network protocol.

2. The care recipient status notification device of claim 1, wherein
each of said input buttons send a signal to the control circuit that translates to an operational command to cause the server to transmit the status message to the computing device when pressed.

3. The care recipient status notification device of claim 2, wherein
in response to receiving the signal, the control circuit is configured to transmit, via the communication device, the operational command to the server to thereby cause the server to transmit the status message to the computing device.

4. The care recipient status notification device of claim 3, wherein
the status message reflects a status of the care recipient and comprises at least one of a:
a pain management assistance request;
an physical comfort assistance request;
a hydration assistance request; and
a toileting assistance request.

5. The care recipient status notification device of claim 4, wherein
the computing device is associated with a non-medical person selected by the care recipient to perform a non-medical task for the care recipient; and
the non-medical task is associated with the status message.

6. The care recipient status notification device of claim 5, wherein
the communications device is configured to communicate with the server via a cellular network communications protocol.

7. The care recipient status notification device of claim 6, wherein
the control circuit is configured to receive a first confirmation signal from the server responsive to the server receiving, from the computing device, a second confirmation signal indicating performance of the non-medical task.

8. The care recipient status notification device of claim 7, wherein
the server is configured to transmit the status message to a second computing device upon expiration of a predetermined time period during which the second confirmation signal is not received by the server.

9. A system for notifying a status of a care recipient, comprising:
a care recipient status notification (CRSN) device;
a care giver computing device;
a server;
wherein
the server
is communicatively coupled to the CRSN device and the care giver computing device;
is configured to communicate via a cellular network protocol;
the CRSN device
is registered to and used by a specific care recipient;
only capable of communicating with the server;
comprises a plurality of input buttons each associated with an input signal that is unique thereto;
each input signal is associated with a status message that is unique thereto; and
the CRSN device is configured to cause the server to transmit the status message to the care giver computing device when it receives the input signal, the status message is a text message.

10. The system of claim 9, wherein
each of said input buttons send a signal to a control circuit of the CRSN device that translates to an operational command to cause the server to transmit the status message to the computing device when pressed.

11. The system of claim 10, wherein
in response to receiving the signal, the CRSN device is configured to transmit the operational command to the server to thereby cause the server to transmit the status message to the computing device.

12. The system of claim 11, wherein
the status message reflects a status of the care recipient and comprises:
a pain management assistance request;
an physical comfort assistance request;
a hydration assistance request; and
a toileting assistance request.

13. The system of claim 12, wherein
the computing device is associated with a non-medical person selected by the care recipient to perform a non-medical task for the care recipient; and
the non-medical task is associated with the status message.

14. The system of claim 13, wherein
the CRSN device is configured to communicate with the server via a cellular network communications protocol.

15. The system of claim 14, wherein
the CRSN device is configured to receive a confirmation signal from the server responsive to the server receiving, from the computing device, a second confirmation signal indicating performance of the non-medical task.

16. The system of claim 15, wherein
the server is configured to transmit the status message to a second computing device upon expiration of a predetermined time period during which the second confirmation signal is not received by the server.
